(19) United States
(12) Patent Application Publication
(10) Pub. No.: US 2025/0090206 A1
(43) Pub. Date: Mar. 20, 2025

(54) Acromial Plate

(71) Applicant: Anup A. Shah, Phoenix, AZ (US)

(72) Inventor: Anup A. Shah, Phoenix, AZ (US)

(21) Appl. No.: 18/368,690

(22) Filed: Sep. 15, 2023

(51) Int. Cl. A61B 17/80

(52) U.S. Cl. CPC A61B 17/8061; A61B 17/8057

(57) ABSTRACT

An acromial plate for use with an acromion, comprising: an acromial support structure having a superior surface, inferior surface, and a posterior edge, the superior surface of the acromial support structure conforming to the inferior surface of the acromion and having at least one screw hole therethrough for receiving a screw, the trajectory of the screw hole therethrough the acromion; an extension having at least one screw hole therethrough for receiving a screw therethrough, the extension extending along at least one surface of the clavicle, the trajectory of the screw hole oriented to project therethrough a clavicle, whereby the acromial support structure buttresses the acromion.

### CROSS-REFERENCE TO RELATED APPLICATIONS

None

### STATEMENT REGARDING FEDERALLY SPONSORED RESEARCH

Not Applicable.

### TECHNICAL FIELD OF THE INVENTION

An acromial plate for treating and preventing or minimizing fractures of the acromion. It is currently known that acromial stress reaction and fractures are a potential complication of reverse total shoulder arthroplasty and other etiologies.

Shoulder injuries are the third most common musculoskeletal complaint. In raw numbers, shoulder injuries are only surpassed by complaints of lower back pain and neck pain. Most shoulder injuries are treated non-operatively. Fractures of the acromion represent a significant danger for patients potentially requiring surgical intervention.

### BACKGROUND OF THE INVENTION

The present invention prevents, mitigates, and treats acromial fractures. Acromial fractures may result from trauma or may occur due to stress or the acromial fracture may occur post-operatively. Acromial stress reaction and fractures are also recognized as potential complication of reverse total shoulder arthroplasty. The present invention may treat a primary fracture or be used as prophylaxis, i.e. to prevent acromial fractures.

FIG. 1A illustrates a partial cross-sectional view of a typical human's shoulder and also illustrates the structures comprising the shoulder.

FIG. 1B illustrates an X-ray of the typical human's shoulder.

FIG. 1C illustrates the position of the acromion in the human skeleton.

FIG. 2 illustrates a Rockwood classification of acromioclavicular joint injuries. The Rockwood classification illustrates six types of acromial fractures (I, II, III, IV, V, and VI).

### SUMMARY OF THE INVENTION

An acromial plate for use with an acromion, comprising: an acromial support structure having a superior surface, inferior surface, and a posterior edge, the superior surface of the acromial support structure conforming to the inferior surface of the acromion and having at least one screw hole therethrough for receiving a screw, the trajectory of the screw hole therethrough the acromion; an extension having at least one screw hole therethrough for receiving a screw therethrough, the extension extending along at least one surface of a clavicle, the trajectory of the screw hole oriented to project therethrough the clavicle, whereby the acromial support structure buttresses the acromion.

These and other embodiments will be more fully appreciated from the description below.

### BRIEF DESCRIPTION OF THE DRAWINGS

FIG. 1A illustrates a typical acromion and its location in a human's shoulder.

FIG. 1B illustrates an x-ray of a typical human shoulder also illustrating a scapula, a clavicle, a coracoid process, and a glenoid.

FIG. 1C illustrates the position of the acromion in the human skeleton.

FIG. 2 illustrates a Rockwood classification of acromioclavicular joint injuries.

FIG. 3 illustrates an anterior view of the acromial plate fastened to the clavicle.

FIG. 4A illustrates a perspective view of an alternative embodiment of the acromial plate fastened to the acromion and a clavicle.

FIG. 4B illustrates a superior view of the alternative embodiment of the acromial plate illustrated in FIG. 4A.

FIG. 5A illustrates a perspective view of a second alternative embodiment of the acromial plate.

FIG. 5B illustrates a perspective view of an alternative embodiment of the acromial plate fastened to the acromion and the scapula.

FIG. 6A illustrates a perspective view of a third alternative embodiment of the acromial plate fastened to the acromion, scapula, and the scapular spine.

FIG. 6B illustrates a perspective view of the alternative embodiment of the acromial plate illustrated in FIG. 6A.

FIG. 6C illustrates a perspective inferior view of the alternative embodiment of the acromial plate illustrated in FIGS. 6A and 6B before attachment.

FIG. 6D illustrates a perspective inferior view of the alternative embodiment of the acromial plate illustrated in FIGS. 6A, 6B, and 6C.

FIG. 6E illustrates a perspective inferior view of a fourth alternative embodiment of the acromial plate.

FIG. 6F illustrates a perspective inferior view of a fifth alternative embodiment of the acromial plate.

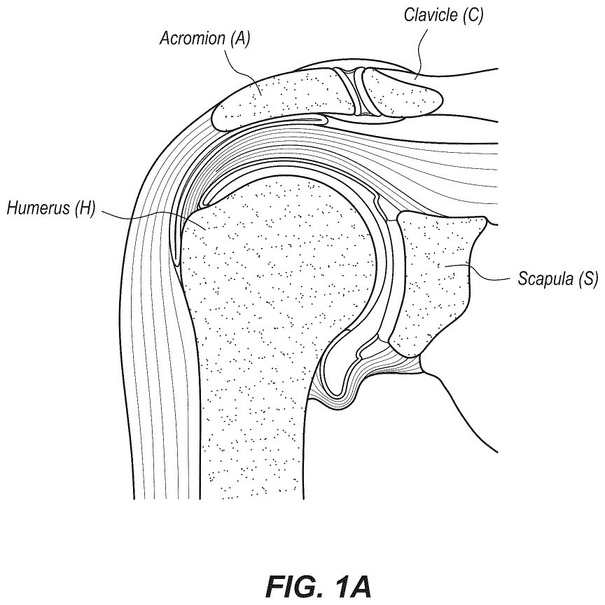

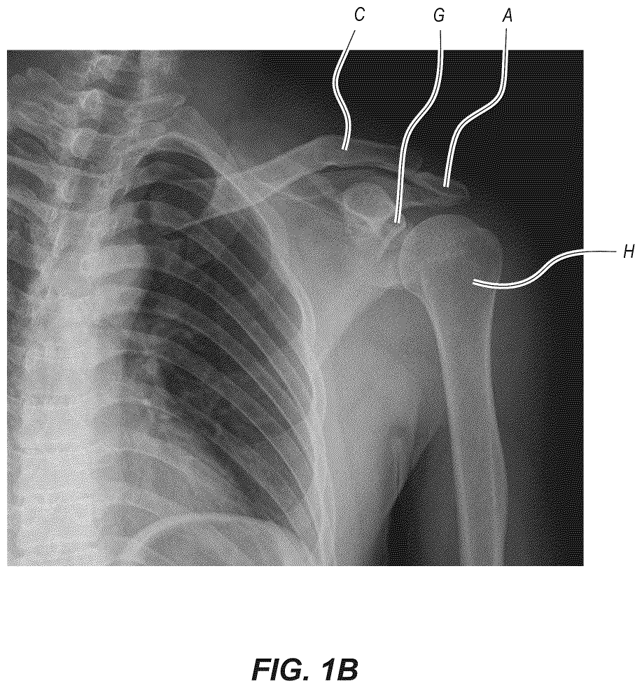

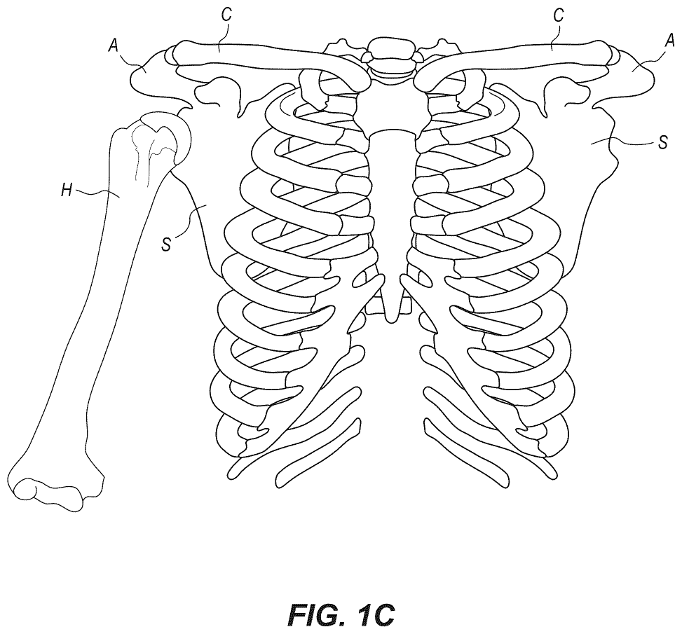

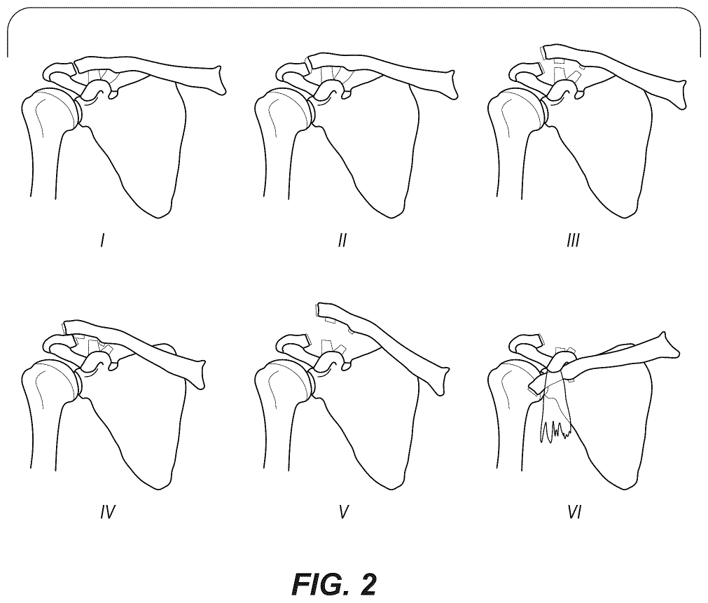

### DESCRIPTION OF THE PREFERRED EMBODIMENTS

Corresponding reference numbers indicate corresponding parts throughout the several views of the drawings and specification.

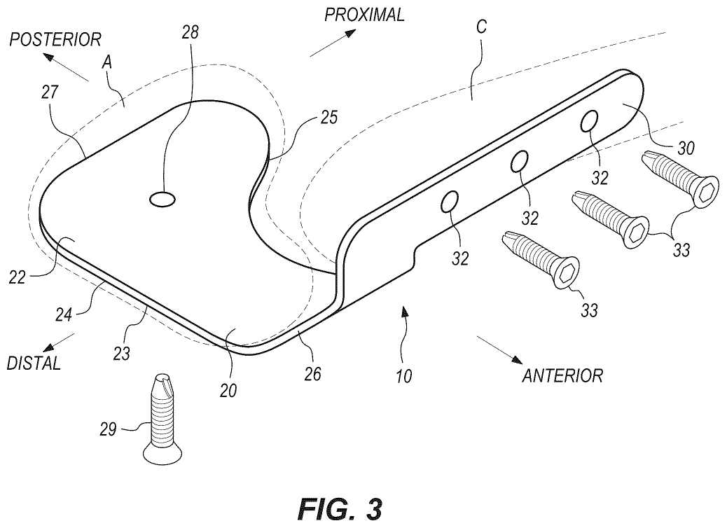

FIG. 3 illustrates an acromial plate 10 for use with an acromion A. The acromial plate 10 having an acromial support structure 20 having a superior surface 22, inferior surface 23, a distal edge 24, a medial edge 25, an anterior edge 26, and a posterior edge 27. The superior surface 22 conforming to the inferior surface of the acromion A and having at least one screw hole 28 therethrough for receiving a screw 29, the trajectory of the screw hole 28 is, preferably, an inferior to superior orientation. Alternatively, screw hole 28 might be thought of as having a superior to inferior trajectory. However, no specific trajectory is required and any trajectory that permits fixation of acromial support structure 20 to the acromion A is appropriate. The acromial support structure 20 having an extension 30 having at least one screw hole 32 therethrough for receiving a screw 33 therethrough. The extension 30 extending along at least one surface of the clavicle C, the trajectory of the screw hole 32 oriented to project therethrough a clavicle C. The acromial support structure 20, and extension 30 buttress the acromion A and treat and prevent acromial fractures. The embodiment of FIG. 3 illustrate the extension 30 fixed to an anterior surface of clavicle C.

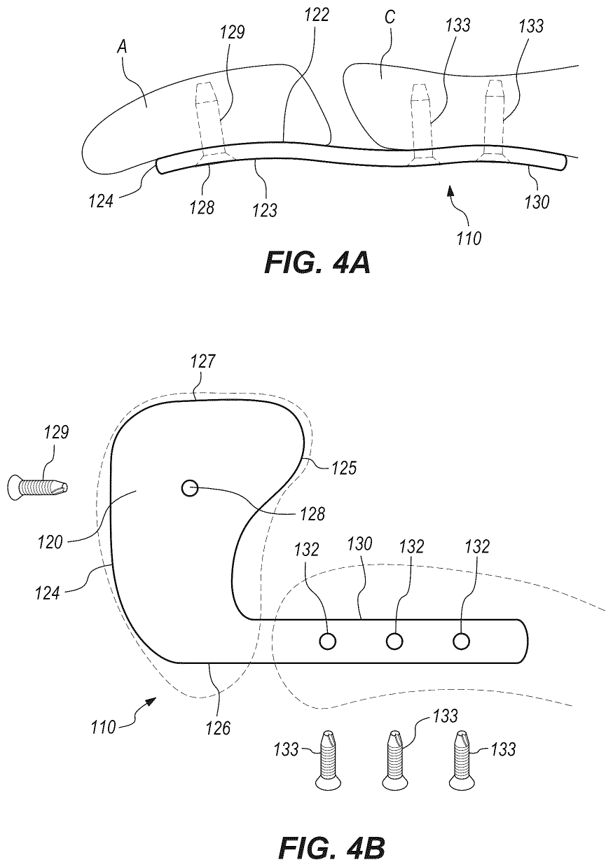

FIGS. 4A and 4B illustrate an acromial plate 110 for use with an acromion A. The acromial plate 110 having an acromial support structure 120 having a superior surface 122, inferior surface 123, a distal edge 124, a medial edge 125, an anterior edge 126, and a posterior edge 127. The superior surface 122 conforming to the inferior surface of the acromion A and having at least one screw hole 128 therethrough for receiving a screw 129, the trajectory of the screw hole 128 is an inferior to superior orientation. Alternatively, screw hole 128 might be thought of as having a superior to inferior trajectory. However, no specific trajectory is required and any trajectory that permits fixation of acromial plate 110 to the acromion A is appropriate. The acromial support structure 120 having an extension 130 having at least one screw hole 132 therethrough for receiving a screw 133 therethrough. The extension 130 extending along at least one surface of the clavicle C, the trajectory of the screw hole 132 oriented to project therethrough a clavicle C. The acromial support structure 120, and extension 130 buttress the acromion A and treat and prevent acromial fractures. The embodiment of FIGS. 4A and 4B illustrates the extension 130 fixed to the inferior surface of clavicle C.

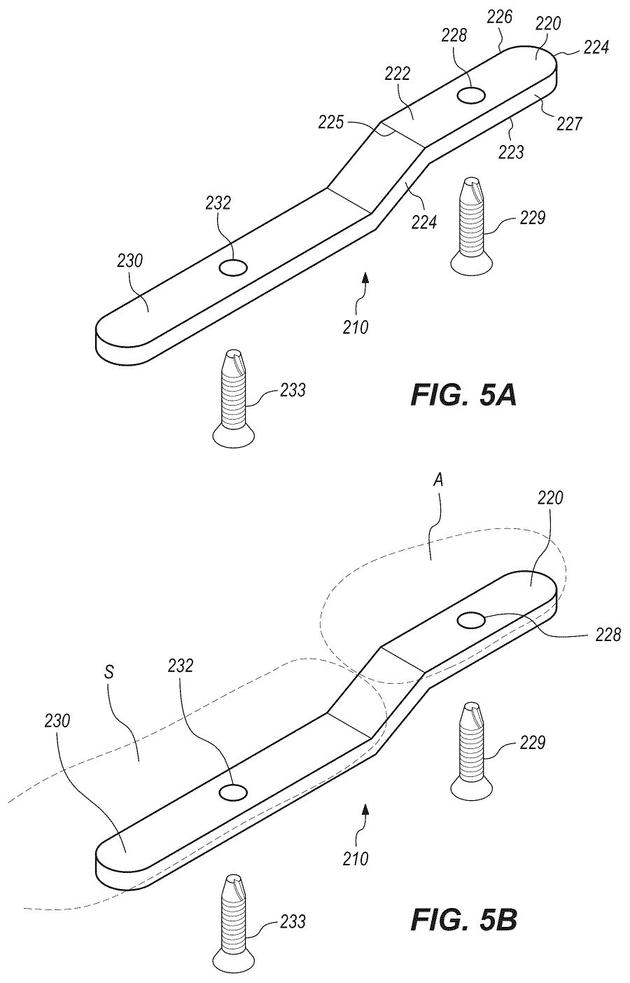

FIGS. 5A and 5B illustrate an acromial plate 210 for use with an acromion A. The acromial plate 210 having an acromial support structure 220 having a superior surface 222, inferior surface 223, a distal edge 224, a medial edge 225, an anterior edge 226, and a posterior edge 227. FIG. 5B illustrates the superior surface 222 conforming to the inferior surface of the acromion A and having at least one screw hole 228 therethrough for receiving a screw 229, the trajectory of the screw hole 228 is an inferior to superior orientation. Alternatively, screw hole 228 might be thought of as having a superior to inferior trajectory. However, no specific trajectory is required and any trajectory that permits fixation of acromial plate 210 to the acromion A is appropriate. The acromial support structure 220 having an extension 230 having at least one screw hole 232 therethrough for receiving a screw 233 therethrough. The extension 230 extending along at least one surface of the scapula S, the trajectory of the screw hole 232 oriented to project therethrough scapula S. The acromial support structure 220, and extension 230 buttress the acromion A and treat and prevent acromial fractures. The embodiment of FIGS. 5A and 5B illustrates the extension 230 fixed to the posterior surface of scapula S.

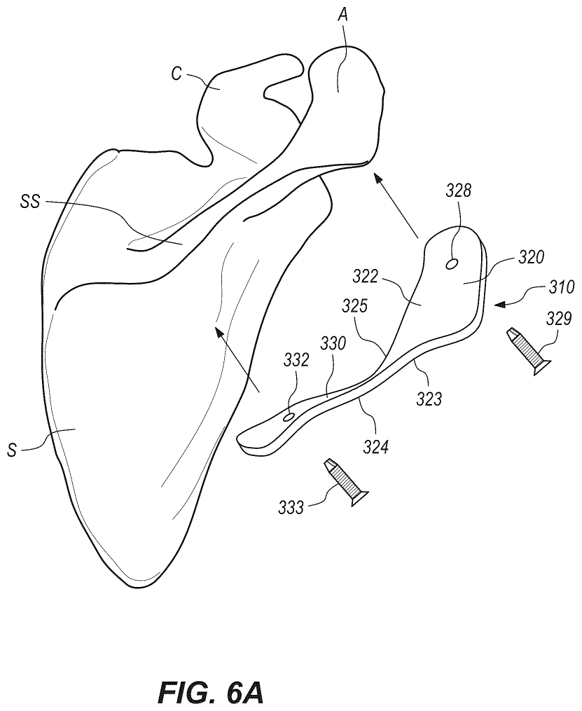

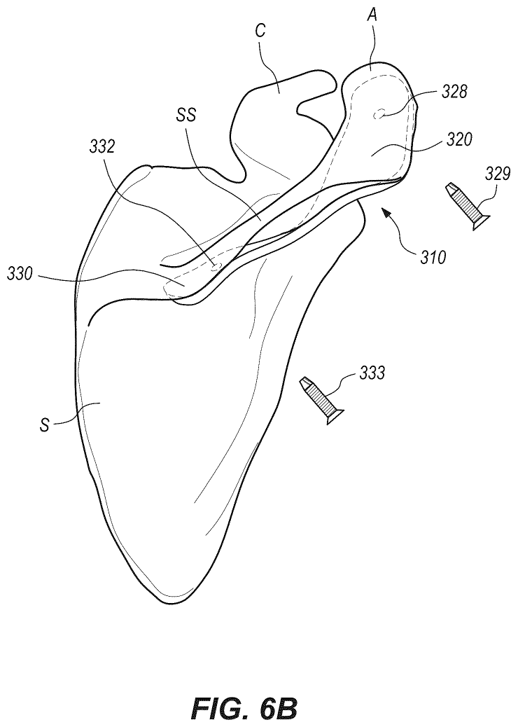

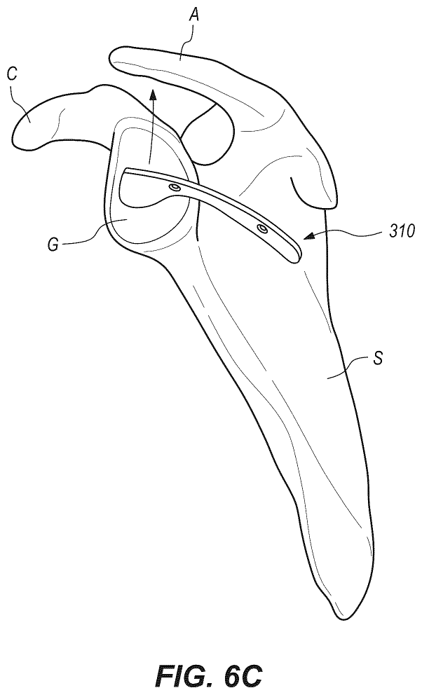

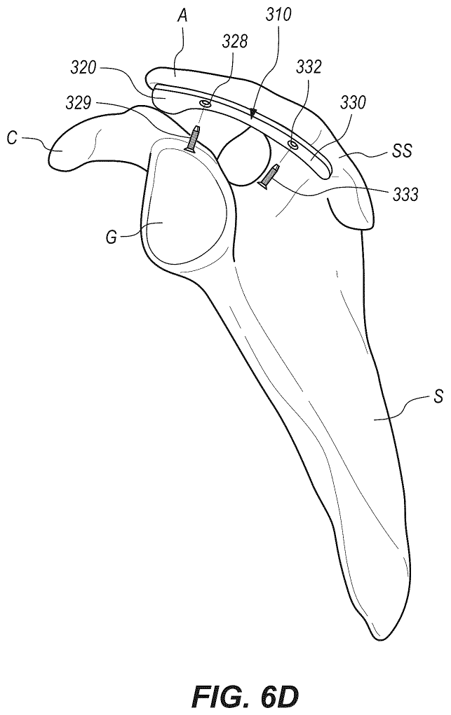

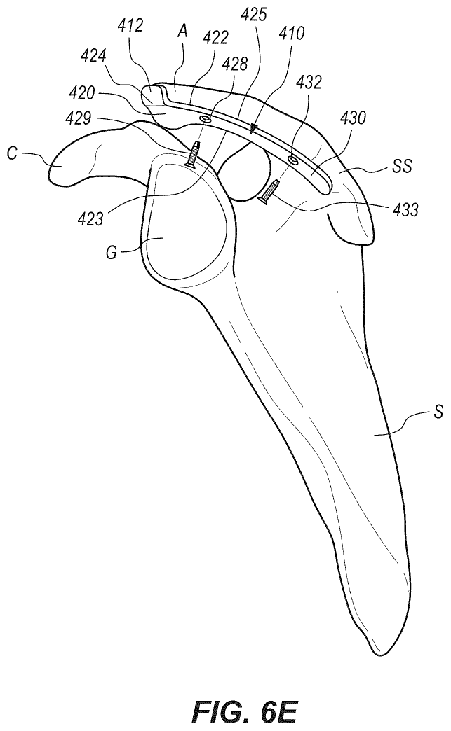

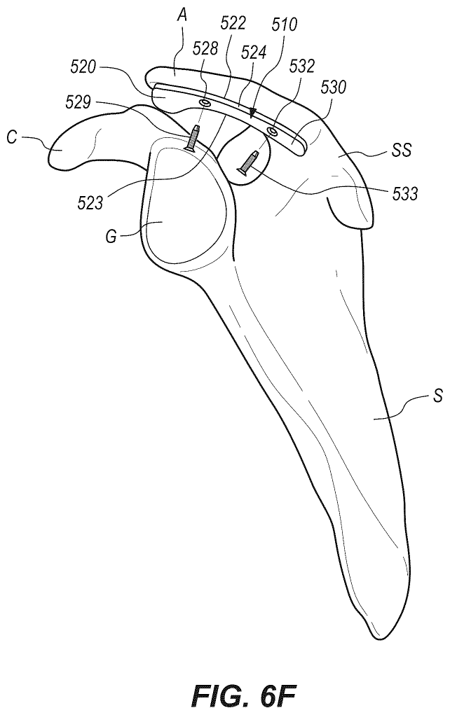

FIGS. 6A, 6B, 6C, and 6D illustrate an acromial plate 310 for use with an acromion A. The acromial plate 310 having an acromial support structure 320 having a superior surface 322, inferior surface 323, a distal edge 324, a medial edge 325. FIGS. 6A, 6B, 6C, and 6D illustrate the superior surface 322 conforming to the inferior surface of the acromion A and having at least one screw hole 328 therethrough for receiving a screw 329, the trajectory of the screw hole 328 is an inferior to superior orientation. Alternatively, screw hole 328 might be thought of as having a superior to inferior trajectory. However, no specific trajectory is required and any trajectory that permits fixation of acromial plate 310 to the acromion A is appropriate. The acromial support structure 320 having an extension 330 having at least one screw hole 332 therethrough for receiving a screw 333 therethrough. The extension 330 extending along the inferior surface of the scapular spine SS of scapula S, the trajectory of the screw hole 332 oriented to project therethrough scapula spine SS. The embodiment of FIG. 6D illustrates the extension 330 fixed to the inferior surface of the scapula spine SS of scapula S. Preferably, extension 330 conforms to the underside of scapular spine SS. The acromial support structure 320, and extension 330 buttress the acromion A and treat and prevent acromial fractures.

FIG. 6E illustrates an acromial plate 410 for use with an acromion A. The acromial plate 410 having an acromial support structure 420 having a superior surface 422, inferior surface 423, a distal edge 424, a medial edge 425 (not visible). The embodiment of FIG. 6E illustrates a lip 412. Lip 412 is preferably integral with plate 410. Lip 412 extends around the edge of acromion A and minimizes or prevents plate 410 being inaccurately placed on the inferior side of acromion A. Lip 412 may be thought of as a lip or “shoe” that prevents or mitigates plate 410 from sliding along acromion A such that plate 412 does not fully coincide with acromion A, i.e., the inferior surface of acromion A fully contacts the superior surface of plate 410. Plate 410 buttress the acromion A and treats and prevent acromial fractures. Preferably, lip 412 may extend along the anterior and posterior edges of the acromion. Plate 410 having at least one screw hole 428 therethrough for receiving a screw 429, the trajectory of the screw hole 428 is an inferior to superior orientation. Alternatively, screw hole 428 might be thought of as having a superior to inferior trajectory. However, no specific trajectory is required and any trajectory that permits fixation of acromial plate 410 to the acromion A is appropriate. The acromial support structure 420 having an extension 430 having at least one screw hole 432 therethrough for receiving a screw 433 therethrough.

FIG. 6F illustrates an acromial plate 510 for use with an acromion A. The acromial plate 510 having an acromial support structure 520 having a superior surface 522, inferior surface 523, a distal edge 524, a medial edge 525 (not visible). FIG. 6F illustrates the superior surface 522 conforming to the inferior surface of the acromion A and having at least one screw hole 528 therethrough for receiving a screw 529, the trajectory of the screw hole 528 is an inferior to superior orientation. Alternatively, screw hole 528 might be thought of as having a superior to inferior trajectory. However, no specific trajectory is required and any trajectory that permits fixation of acromial plate 510 to the acromion A is appropriate. FIG. 6F illustrates that acromial support surface 520 might only extend partially along acromion A, i.e., that plate 510 supports only a portion of acromion A. However, whatever portion of the acromion A is supported, the acromial support structure 520 buttresses the acromion and treats and prevents acromial fractures.

Preferably, any extension (e.g. 130, 230, 330, 430, 530) is integral with the other elements of the acromial plate. Fixation of the acromial plate is preferably accomplished with screws, pins, buttons, sutures, or the like. Installation of the acromial plate is preferably accomplished using a lateral incision or an anterior incision to position the plate on the inferior surface of the acromion A.

There are three morphologically distinct types of acromia: flat, curved and hooked. Thus, in many humans, the inferior surface of the acromion is not flat. In the preferred embodiment, the inferior surface of the acromial support structure would be modeled and the superior surface of the acromial support structure would be prepared to conform to the inferior surface of the acromion. An example of a device used to model the inferior surface of the acromion is the 3-D computer tomography scanner, 320-slice CT Scanner, Aquilon One by Toshiba.

Preferably, the acromial plates can be fixed to the acromion A and scapula S, scapular spine of the scapula SS, or clavicle C by a screw, button, suture, or other fixation to fix, i.e. attach/fasten metal to bone. Particularly preferably, the screw or other fixation device should be flush with the bone.

While the invention has been illustrated and described in detail in the drawings and description, the same is to be considered as an illustration and is not limited to the exact embodiments shown and described. All equivalents, changes and modifications that come within the spirit of the invention are also protected by the claims that are set forth below.

What I claimed is:

1. An acromial plate for use with an acromion, comprising:
an acromial support structure having a superior surface and an inferior surface, the superior surface of the acromial support structure conforming to the inferior surface of the acromion and having at least one screw hole therethrough for receiving a screw, the trajectory of the screw hole therethrough the acromion;
an extension having at least one screw hole therethrough for receiving a screw therethrough, the extension extending along at least one surface of a clavicle, the trajectory of the screw hole oriented to project therethrough the clavicle,
whereby the acromial support structure buttresses the acromion.

2. The acromial plate of claim 1, further comprising: the extension extends along an anterior side of the clavicle.

3. The acromial plate of claim 1, further comprising: the extension extends along an inferior side of the clavicle.

4. The acromial plate of claim 1, further comprising: the extension extends along either the inferior or the anterior side of the clavicle.

5. The acromial plate of claim 1, further comprising: a screw for fixing the acromial support structure to the acromion and another screw for fixing the extension to the clavicle.

6. The acromial plate of claim 5, further comprising: the screws are screws, buttons, or sutures.

7. An acromial plate for use with an acromion, comprising:
an acromial support structure having a superior surface and an inferior surface, the superior surface of the acromial support structure conforming to the inferior surface of the acromion and having at least one screw hole therethrough for receiving a screw, the trajectory of the screw hole therethrough the acromion;
an extension having at least one screw hole therethrough for receiving a screw therethrough, the extension extending along at least one surface of the scapula, the trajectory of the screw hole oriented to project therethrough the scapula,
whereby the acromial support structure buttresses the acromion.

8. The acromial plate of claim 7, further comprising: the extension extends along the scapula.

9. The acromial plate of claim 8, further comprising: the acromial support structure extends only partially along the acromion or scapula.

10. The acromial plate of claim 8, further comprising: the extension extends along a posterior surface of the scapula.

11. The acromial plate of claim 7, further comprising: a screw for fixing the acromial support structure to the acromion and another screw for fixing the extension to the scapula.

12. The acromial plate of claim 11, further comprising: the screw for fixing the acromial support structure to the acromion and the another screw for fixing the extension to the scapula are screws, buttons, or sutures.

13. The acromial plate of claim 7, further comprising: the extension extends along an inferior surface of a scapular spine of the scapula.

14. The acromial plate of claim 13, further comprising: a screw for fixing the acromial support structure to the acromion and another screw for fixing the extension to the scapula.

15. An acromial plate for use with an acromion, comprising:
an acromial support structure having a superior surface and an inferior surface, the superior surface of the acromial support structure conforming to the inferior surface of the acromion and having at least one screw hole therethrough for receiving a screw, the trajectory of the screw hole extending therethrough the acromion;
an extension having at least one screw hole therethrough for receiving a screw therethrough, the extension extending along at least one surface of the scapula and a scapular spine of the scapula, the trajectory of the screw hole oriented to project therethrough the scapula,
whereby the acromial support structure buttresses the acromion.

16. The acromial plate of claim 15, further comprising: the extension extends along an inferior surface of the scapular spine of the scapula.

17. The acromial plate of claim 16, further comprising: a screw for fixing the acromial support structure to the acromion and another screw for fixing the extension to the scapula.

18. The acromial plate of claim 17, further comprising: a screw for fixing the acromial support structure to the scapula spine of the scapula.

19. The acromial plate of claim 17, further comprising: the screw for fixing the acromial support structure is a screw, a button, or a suture.

20. The acromial plate of claim 18, further comprising: the screw for fixing the extension to the scapular spine of the scapula is a screw, a button, or a suture.
